(19) United States
(12) Patent Application Publication
(10) Pub. No.: US 2008/0020060 A1
(43) Pub. Date: Jan. 24, 2008

(54) PROCESS FOR TREATING RHINITIS

(76) Inventor: Robert R. Williamson, Dallas, TX (US)

Correspondence Address: Michael A. O'Neil;Michael A. O'Neil, P.C., Suite 820, 5949 Sherry Lane, Dallas, TX, 75225, US

(21) Appl. No.: 11/768,261

(22) Filed: Jun. 26, 2007

Related U.S. Application Data
(60) Provisional application No. 60/820,003, filed on Jul. 21, 2006.

(51) Int. Cl. A61K 33/38; A61P 31/00; A61P 31/16

(52) U.S. Cl. 424/618

(57) ABSTRACT

A colloidal suspension comprising elemental silver and/or one or more alloys of silver is mixed with either an over-the-counter rhinitis medicine or a prescription rhinitis medicine. The resulting mixture is administered to rhinitis patients in the form of a spray.

### CROSS-REFERENCE TO RELATED APPLICATION

Applicant claims priority based on provisional patent application Ser. No. 60/820,003 filed Jul. 21, 2006, the entire content of which is incorporated herein by reference.

### TECHNICAL FIELD

This invention relates generally to the treatment and control of rhinitis, and more particularly to the use of silver and silver alloys in the treatment and control of rhinitis.

### BACKGROUND AND SUMMARY OF THE INVENTION

Rhinitis has adversely affected humankind literally for centuries, and even more so since the advent of the industrial age. More recently the existence of a multitude of airborne allergens, pathogens, and irritants has resulted in substantial portions of the populations of all industrialized nations being afflicted to a greater or lesser degree with rhinitis.

Over the centuries a wide variety of homeopathic remedies, patent medicines, over-the-counter drugs, and prescription drugs have been available for the treatment of rhinitis. Despite the almost universal availability of existing rhinitis treatments, widespread control of rhinitis symptoms has not yet been achieved. Thus, a need exists for a method of treating rhinitis which is both effective and readily available.

The anti-microbial benefits of silver have long been recognized. For example, from ancient times through the present people have recognized the fact that foods and beverages are less likely to spoil when stored, prepared, and served in silver cooking utensils, serving utensils, and beverage containers.

In accordance with the present invention elemental silver and/or alloys of silver are employed in the treatment and control of rhinitis. More specifically, a colloidal suspension comprising de-ionized water and nanoparticles of silver and/or one more alloys of silver is utilized in the treatment of rhinitis.

In accordance with a first embodiment of the invention a colloidal suspension of elemental silver and/or one or more alloys of silver is mixed with a selected over-the-counter rhinitis treatment medicine. The resulting mixture is discharged into the nostrils of a patient in the form of a spray. In most instances a once daily administration of the mixture is sufficient to completely control rhinitis.

In accordance with a second embodiment of the invention a colloidal suspension of elemental silver and/or one or more silver alloys is mixed with a selected prescription rhinitis treatment medicine. The resulting mixture is discharged into the nostrils of a patient in the form of a spray. In most instances a once daily administration of the mixture is sufficient to completely control rhinitis.

### DETAILED DESCRIPTION

### EXAMPLE 1

A colloidal suspension comprising nanoparticles of elemental silver is provided. The colloidal suspension comprises de-ionized water and 99.99% pure silver. The colloidal suspension comprises approximately 20 ppm silver.

The foregoing colloidal suspension comprising substantially pure silver is mixed with an over-the-counter rhinitis remedy. Examples of over-the-counter rhinitis medicines which may be used in the practice of the invention include AFRIN® (oxymetazoline HCl), NEO-SYNEPHRINE® (phenylephrine HCl 1.0%); and DRISTAN® (oxymetazoline HCl 0.05%).

The colloidal suspension of substantially pure elemental silver and the selected over-the-counter rhinitis medicine are mixed at a ratio of 1:1. The resulting mixture is thereafter administered into the nostrils of a rhinitis patient in the form of a spray. It has been found that a once daily administration of the foregoing mixture is sufficient to substantially completely control rhinitis.

### EXAMPLE 2

A colloidal suspension comprising nanoparticles of elemental silver is provided. The colloidal suspension comprises de-ionized water and 99.99% pure silver. The colloidal suspension comprises approximately 20 ppm silver.

The foregoing colloidal suspension comprising substantially pure silver is mixed with a prescription rhinitis remedy. Examples of prescription rhinitis medicines which may be used in the practice of the invention include FLONASE® (fluticasone propionate) NASONEX® (mometasone furoate monohydrate), NASACORT® (triamcinolone acetonide, and BECONTAS AQ® (beclomethasone dipropionate, monohydrate).

The colloidal suspension of substantially pure elemental silver and the selected prescription rhinitis medicine are mixed at ratio of 1:1. The resulting mixture is thereafter administered into the nostrils of a rhinitis patient in the form of a spray. It has been found that a once daily administration of the foregoing mixture is sufficient to substantially completely control rhinitis.

### EXAMPLE 3

A colloidal suspension comprising nanoparticles of one or more alloys of silver is provided. The colloidal suspension comprises de-ionized water and one or more substantially pure silver alloys. The colloidal suspension may comprise approximately 20 ppm of one or more silver alloys. The elements that may be alloyed with silver for use in the practice of the invention include the following: Sn, Zn, Au, Pt, Pd, Co, Rh, Ir, Mn, Ti, and Zr.

The foregoing colloidal suspension comprising substantially one or more alloys of silver is mixed with an over-the-counter rhinitis remedy. Examples of over-the-counter rhinitis medicines which may be used in the practice of the invention include AFRIN® (oxymetazoline HCl), NEO-SYNEPHRINE® (phenylephrine HCl 1.0%), and DRISTAN® (oxymetazoline HCl 0.05).

The colloidal suspension of one or more alloys of silver and the selected over-the-counter rhinitis medicine are mixed at a ratio of 1:1. The resulting mixture is thereafter administered into the nostrils of a rhinitis patient in the form of a spray. It has been found that a once daily administration of the foregoing mixture is sufficient to substantially completely control rhinitis.

### EXAMPLE 4

A colloidal suspension comprising nanoparticles of one or more alloys of silver is provided. The colloidal suspension comprises de-ionized water and one or more substantially pure silver alloys. The colloidal suspension may comprise approximately 20 ppm of one or more silver alloys. The elements that may be alloyed with silver for use in the practice of the invention include the following: Sn, Zn, Au, Pt, Pd, Co, Rh, Ir, Mn, Ti, and Zr.

The foregoing colloidal suspension comprising substantially pure silver is mixed with a prescription rhinitis remedy. Examples of prescription rhinitis medicines which may be used in the practice of the invention include FLONASE® (fluticasone propionate); NASONEX® (mometasone furoate monohydrate), NASACORT® (triamcinolone acetonide), and BECONAS AQ® (beclomethasone dipropionate, monohydrate).

The colloidal suspension of substantially pure elemental silver and the selected prescription rhinitis medicine are mixed at a ratio of 1:1. The resulting mixture is thereafter administered into the nostrils of a rhinitis patient in the form of a spray. It has been found that a once daily administration of the foregoing mixture is sufficient to substantially completely control rhinitis.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of treating rhinitis comprising the steps of:
providing a colloidal suspension of elemental silver;
providing a predetermined over-the-counter rhinitis treatment medicine;
mixing the colloidal suspension of elemental silver with the over-the-counter rhinitis treatment medicine at a predetermined ratio; and
administering the resulting mixture into the nostrils of a rhinitis patient in the form of a spray.

2. The method of treating rhinitis according to claim 1 wherein the colloidal suspension of elemental silver comprises de-ionized water and 20 ppm of 99.9% pure silver.

3. The method of treating rhinitis according to claim 1 wherein the predetermined ratio is 1:1.

4. A method of treating rhinitis comprising the steps of:
providing a colloidal suspension of elemental silver;
providing a predetermined prescription rhinitis treatment medicine;
mixing the colloidal suspension of elemental silver with the prescription rhinitis treatment medicine at a predetermined ratio; and
administering the resulting mixture into the nostrils of a rhinitis patient in the form of a spray.

5. The method of treating rhinitis according to claim 4 wherein the colloidal suspension of elemental silver comprises de-ionized water and 20 ppm of 99.9% pure silver.

6. The method of treating rhinitis according to claim 4 wherein the predetermined ratio is 1:1.

7. A method of treating rhinitis comprising the steps of:
providing a colloidal suspension of one or more alloys of silver;
providing a predetermined over-the-counter rhinitis treatment medicine,
mixing the colloidal suspension of one or more alloys of silver with the over-the-counter rhinitis treatment medicine at a predetermined ratio; and
administering the resulting mixture into the nostrils of a rhinitis patient in the form of a spray.

8. The method of treating rhinitis according to claim 7 wherein at least one of the alloys of silver comprises silver alloyed with an element selected from the group consisting of Sn, Zn, Au, Pt, Pd, Co, Rh, Ir, Mn, Ti, and Zr.

9. The method of treating rhinitis according to claim 7 wherein the predetermined ratio is 1:1.

10. A method of treating rhinitis comprising the steps of:
providing a colloidal suspension of one or more alloys of silver;
providing a predetermined prescription rhinitis treatment medicine;
mixing the colloidal suspension of one or more alloys of silver with the prescription rhinitis treatment medicine at a predetermined ratio; and
administering the resulting mixture into the nostrils of a rhinitis patient in the form of a spray.

11. The method of treating rhinitis according to claim 10 wherein at least one of the alloys of silver comprises silver alloyed with an element selected from the group consisting of Sn, Zn, Au, Pt, Pd, Co, Rh, Ir, Mn, Ti, and Zr.

12. The method of treating rhinitis according to claim 10 wherein the predetermined ratio is 1:1.
